(19) United States
(12) Patent Application Publication
(10) Pub. No.: US 2009/0253589 A1
(43) Pub. Date: Oct. 8, 2009

(54) Method for Testing Active Compounds

(75) Inventors: Daniel Müller, Dresden (DE); Tanuj Sanpra, Dresden (DE); Krzysztof Palczewski, Cleveland, OH (US); Paul Shin-Hyun Park, Cleveland, OH (US)

Correspondence Address: FROST BROWN TODD, LLC, 2200 PNC CENTER, 201 E. FIFTH STREET, CINCINNATI, OH, 45202, US

(73) Assignee: Nambition GmbH, Dresden (DE)

(21) Appl. No.: 12/440,126

(22) PCT Filed: Sep. 5, 2006
(86) PCT No.: PCT/EP2006/008645

§ 371 Date: Jun. 17, 2009

(51) Int. Cl. C40B 30/10; G01N 33/68; C12Q 1/00; C12Q 1/02

(52) U.S. Cl. 506/12; 436/86; 435/4; 435/29

(57) ABSTRACT

In one aspect the invention relates to a method for testing a chemical entity for its capability to modulate a (poly)peptide that is malfunctioning by means of an interaction of said chemical entity and said (poly)peptide, the method using single-molecule force spectroscopy. In another aspect the invention relates to a method for testing a chemical or physical entity for its capability to interact with a G protein-coupled receptor (GPCR) in its natural membrane environment, the method using single-molecule force spectroscopy.

### TECHNICAL FIELD

This invention relates to a method for testing a physical or chemical entity that potentially modulates malfunctioning proteins and to a method for testing such an entity for its capability to interact with a G protein-coupled receptor (GPCR) in its natural membrane surroundings. Particularly, this invention relates to such methods using single-molecule force spectroscopy.

### BACKGROUND OF THE INVENTION

The majority of drugs on the market target either human or pathogen membrane proteins. Additionally there are drugs that target soluble proteins other than membrane proteins. Therefore (poly)peptides and proteins are of outstanding importance in drug screening.

Receptors are the proteins most often targeted by drugs. The classic approach to receptor-based drug design has historically been simplistic. Drug discovery programs have been very successful in identifying compounds that interact with the orthosteric site on a receptor—that compete with the endogenous agonist either to activate or to block the activation of the receptor. The lead compound is usually identified by screening libraries of various candidate compounds and characterizing it biochemically to give K_{d} (dissociation or association constants), which denotes the affinity of the compound to the active site of the receptor.

However, in many cases a more subtle modulation of the receptor is desirable rather than its complete inactivation or activation. For example, the receptor activity could be made more sensitive or less sensitive to the native ligand by tinkering the conformational states of the receptor. This can be achieved allosterically by using drugs that do not bind to the major native ligand binding site of the receptor but to another site, thus indirectly altering the activity. It is known that in many cases of malfunctioning an aberrant conformation of a protein is involved.

In the above cases where there is an interaction between a ligand and a receptor the receptor is necessarily present and usually works at least to a certain degree. There are, however, some diseases where the receptor molecule folds incorrectly, e.g. because of a point mutation, and fails to be correctly incorporated in the membrane to give the final functional molecule. Approaches to remedy these cases use the so-called “chaperone” molecules that induce the receptor to fold in a way in which a normal receptor molecule would fold. The receptor is thus incorporated into the membrane where it might work at least partially, even if it doesn't achieve the native fold, in a way a normal receptor would function.

The most abundant receptors in the body are G protein-coupled receptors (GPCRs) that fall into three classes depending on the respective G protein subunits, i.e. Gₛ, G_{q} and G_{i/o}, involved. All GPCRs have seven transmembrane helices that extend from the extracellular space to the cytosol of the cell. GPCRs transmit signals induced by the binding of a ligand from the exterior of the cell via the seven transmembrane helices to the interior of the cell where they are processed according to the GPCR class.

GPCRs represent one of the most important classes of drug targets, with an estimated 60% of currently marketed drugs directed against them [1]. However, the development of allosteric modulator drugs for GPCRs has just recently begun. One approved and registered example is cinacalcet, which is an allosteric modulator of the parathyroid calcium receptor leading to an enhanced sensitivity of the receptor for calcium and to a reduced release of parathyroid hormone. It is used in the therapy for secondary hyperparathyroidism.

Most cases of GPCR-related diseases involve receptor malfunctioning. However, there are cases where the receptor molecules are not even incorporated into the membrane because of misfolding due to a mutation. This is the case with certain mutants of the rhodopsin receptor in the eye causing the disease retinitis pigmentosa which eventually leads to vision loss. Other examples are V₂, vasopressin receptor (V₂R) mutants responsible for nephrogenic diabetes insipidus. With some mutants chaperone molecules, which are either agonists or antagonists of the receptor, lead to a rescue of cell surface expression (near native folding and insertion into the membrane) and function.

It is evident that a high throughput method of effectively screening positive lead compounds, e.g. an allosteric modulator of malfunctioning GPCRs or a chaperone molecule for misfolded GPCRs, by detecting changes in the conformation of a protein induced by the compounds would be of a great value.

Force spectroscopy is a novel technique that allows the study of mechanical and physical properties of chemical bonds in a variety of systems, such as ligand/receptor pairs or single polymeric molecules. When applied to single molecules the method is called single-molecule force spectroscopy (SMFS). SMFS measures the behaviour of a single molecule under stretching or torsional mechanical force.

Common applications of force microscopy are measurements of polymer elasticity, especially of biopolymers such as RNA and DNA, and unfolding of water-soluble proteins, as well as the mechanical resistance of chemical bonds, for example in antigen-antibody complexes.

There are a number of methods available to accurately manipulate single molecules. The most commonly used are laser optical or magnetic tweezers and atomic-force-microscope (AFM). Whereas the force gradient of an electromagnetic field is used to apply force in optical or magnetic tweezers, a rectangular or beam shaped cantilever is the “hand” of AFM that applies a force. In a nutshell, to measure the mechanical properties of a molecule, one end of the molecule is tethered to a support and the other end pulled using a cantilever. The force applied by or acting on the cantilever over a certain distance can be easily determined by simple physical conversions.

In 1982 G. Binnig and G. Rohrer developed the so-called “Scanning Tunneling Microscope” at the IBM research laboratory in Zurich to study conducting samples. The subsequent development of the “Scanning Force Microsope” or “Atomic Force Microscope” in 1985 by G. Binning together with C. F. Quate, also allowed the investigation of insulating and semiconducting samples.

In the atomic force microscope a sharp tip attached to an elastic cantilever scans over a surface. In principle an atomic force microscope works like a record player where a needle is scanned over an uneven surface and the changes in the level of the needle generates an electric signal. The movement of the tip and the cantilever is detected by means of a laser, by interferometry, by means of capacitance or more recently by a photodiode. Compared to conventional light microscopy particularly in biology, atomic force microscopy has the advantages of achieving molecular resolution and the capability to be carried out under almost native conditions in liquids.

Since 1990 the tip of an AFM cantilever has served as nanotweezers, enabling to manipulate biological or chemical objects at the molecular scale [2, 3, 4, 5, 6]. One end of a single polymeric molecule can be adsorbed to the AFM tip, the other end being attached e.g. by chemical, physical or biological interactions, on a supporting surface. The cantilever can subsequently be separated from the surface by moving a piezoelectric actuator/transducer. This separation then unravels the polymeric molecule. At a certain separation the molecule is stretched between the support and the tip and a force acts on the elastic cantilever. This force deflects the cantilever upward (repulsive force) or downward (attractive force): According to Hooke's law, this deflection will be proportional to the force acting on the cantilever. By this technique forces as low as a few pN (10⁻¹² N) can be measured. Only thermal noise limits the measurement. The so-called force curve is the graph of force (or more precisely, the cantilever deflection) versus the piezoelectric position on the z-axis. An ideal Hookean spring, for example, would display a straight diagonal force curve, whereas the sequential unfolding of proteins exhibits a very characteristic sawtooth pattern of the force vs. elongation graph wherein every tooth corresponds to the unfolding of one specific structural segment of the protein.

The outstanding positioning precision (≈0.1 nm) and force sensitivity (≈5 pN) of the AFM has made even the most delicate single-molecule unfolding experiments possible using force spectroscopy. In these experiments, the force applied to a single protein plays the role of a denaturant leading to complete unfolding of its three-dimensional structure. In their initial studies, Rief and co-workers applied single-molecule force spectroscopy to the giant muscle protein titin, which consists of repeats of globular immunoglobulin and fibronectin domains [5,7]. The continuous extension of the protein resulted in the subsequent unfolding of the globular domains allowing the unfolding force and pathway of each domain to be detected [7,8,9].

In contrast to many experiments performed on water-soluble proteins, the application of single-molecule force spectroscopy to membrane proteins [45,46] yielded surprisingly detailed insights into the inter- and intramolecular interactions stabilizing their three-dimensional structure. This has been demonstrated on membrane proteins like bacteriorhodopsin (BR) [10,11], halorhodopsin [12] from Halobacterium salinarum, human aquaporin-1 [13], and the Na⁺/H+ antiporter NhaA from Escherichia coli [14]. To select a membrane protein for a force spectroscopy experiment the protein containing membrane was first imaged at sub-nanometer resolution. Then AFM tip and the selected protein are brought into contact. Applying a force of 300-1000 pN results in binding of one terminal end to the tip either via a covalent bond [15] or enforced non-specific adsorption [16]. Withdrawing the tip from the membrane stretches the terminus of the protein and causes the cantilever to deflect. Upon further separating the tip and surface, the force pulling on the protein steadily increases. As soon as the force exceeds the stability of the protein it induces the sequential unfolding of its three-dimensional structure. Recording the force against tip-surface separation yields a force-distance spectrum characteristic of the unfolding of a single protein. The presence of several distinct events in the force spectrum indicates that secondary structure elements of membrane proteins unfold in well-defined sequences. As their characteristic saw-tooth pattern stems from the extension of already unfolded polypeptide elements, the unfolding spectra are readily analyzed with the wormlike chain (WLC) model [17].

Single-molecule force spectroscopy (SMFS) [18,19,20] provides novel approaches to characterize water-soluble and membrane proteins under variable physiological environments [21,22,23]. In all measurements the proteins were exposed to buffer solution at ambient temperatures. It was shown in several examples that single-molecule force spectroscopy enables to detect inter- and intramolecular interactions within and between proteins [7,15,24,25]. Such experiments not only enabled to detect the stability of membrane proteins [26], but also to probe their energy landscape [27] and refolding kinetics [14]. Single potential barriers confine structurally stable segments that may be represented by transmembrane α-helices, polypeptide loops or fragments thereof. These structural segments are established by collective interactions of several amino acids. Once the externally applied force overcomes the stability of these segments they unfold spontaneously. The first experiments allowed investigating how environmental variations such as the oligomeric assembly, temperature changes, point mutations, or pH variations influenced the stability of these structural segments and thereby the unfolding pathways of the protein. Comparing structurally stable segments established within two different membrane proteins having almost identical structures allowed to gain insights into the origin of these interactions [12]. Recently, it has become possible to observe the refolding of secondary structure elements into the final protein and to estimate its folding kinetics from single-molecule experiments [14].

In our earlier application “Method for Determining the State of Activation of a Protein” (PCT/EP2006/004492, filed on 13 May 2005, and US Provisional Application, filed on 11 Oct. 2005) which is incorporated herein in its entirety by reference, we detected and located molecular interactions that activate a protein. It could be shown at which location a ligand binds to the protein and thereby activates the protein function. Monitoring the activation state of a protein permitted to test the effect of an activator or inhibitor on the functional state of the protein.

### SUMMARY OF THE INVENTION

In first broad aspect the invention relates to a method for testing a chemical entity for its capability to modulate a (poly)peptide that is malfunctioning by means of an interaction of said chemical entity and said (poly)peptide, said method using single-molecule force spectroscopy.

In one aspect the invention relates to a method for testing a chemical entity for its capability to modulate a (poly)peptide that is malfunctioning via an interaction of said chemical entity and said (poly)peptide, comprising the steps of
(a) immobilizing said malfunctioning (poly)peptide on a carrier;
(b) attaching said (poly)peptide immobilized on said carrier to a force measuring device of a force spectroscope;
(c) applying a pulling force to said (poly)peptide attached to said force measuring device and measuring the force required for stretching and/or unfolding said (poly)peptide vs. the elongation of said (poly)peptide immobilized on said carrier and attached to said force measuring device, wherein the measurements are carried out (i) prior to and (ii) after treatment of said malfunctioning (poly)peptide with said chemical entity;
(d) optionally comparing a force spectrum of said (poly)peptide obtained prior to said treatment and a force spectrum of said (poly)peptide obtained after said treatment, and, if the spectra are different,
(e) optionally obtaining a force spectrum of a native (poly)peptide untreated with said chemical entity that is correctly functioning and corresponds to said poly(peptide) that is malfunctioning according to steps (a), (b), and (c) (i) above;
(f) optionally comparing the force spectrum of the native (poly)peptide untreated with said chemical entity that is correctly functioning with the force spectra of said (poly)peptide obtained according to steps (c) (i) and (c) (ii) above, and
(g) optionally concluding from the difference of the force spectra of step (d) and step (f) whether said chemical entity is modulating the malfunctioning (poly)peptide towards a state of correctly functioning.

In another broad aspect the invention relates to a method for testing a chemical or physical entity for its capability to interact with a G protein-coupled receptor (GPCR) in its natural membrane environment, said method using single-molecule spectroscopy.

In another aspect the invention relates to a method for testing a chemical or physical entity for its capability to interact with a G protein-coupled receptor (GPCR) in its natural membrane environment, the method comprising the steps of
(a) immobilizing said GPCR in its natural membrane environment on a carrier;
(b) attaching said immobilized GPCR on said carrier to a force measuring device of a force spectroscope;
(c) applying a pulling force to said GPCR attached to said force measuring device and measuring the force required for stretching and/or unfolding said GPCR vs. the elongation of said GPCR immobilized on said carrier and attached to said force measuring device, wherein the measurements are carried out (i) prior to and (ii) after treatment of said GPCR with said chemical or physical entity;
(d) optionally comparing a force spectrum of said GPCR obtained prior to said treatment and a force spectrum of said GPCR obtained after said treatment, and
(e) optionally concluding from a difference of the force spectra of (d) whether said physical or chemical entity interacts with said GPCR.

### SHORT DESCRIPTIONS OF THE FIGURES

FIGS. 1 (a) to (c) show single-molecule force spectroscopy curves recorded on native ROS disc membranes; FIG. 1 (d) shows the secondary structure of rhodopsin. 1 (a) Representative force-distance curve reflecting the unfolding of a single Rh molecule. The force curve exhibits a length of ≈65 nm, which corresponds to that of an entirely unfolded rhodopsin molecule with an intact Cys110-Cys187 bond. Individual force peaks reflect molecular interactions that occurred while unfolding the protein. Fitting each force peak with the wormlike chain (WLC) model reveals the number of amino acid residues at which the molecular interactions occurred. The values obtained from the WLC model are shown at the end of each fit. 1 (b) A selection of force-distance curves recorded under identical experimental conditions. A comparison of the force curves reveals that some force peaks occur in the majority of curves (main peaks) and some occur in only a small fraction of curves (minor peaks). Each characteristic force pattern of a curve represents molecular interactions that had to be overcome to unfold the protein. Force curves exhibiting the same major and minor force peaks indicate that the rhodopsin molecule unfolded taking the same pathway. 1 (c) The superimposition of several force curves (n=42) enhanced common features among the curves. Major (black) and minor peaks (grey shaded) were fitted using the WLC model as described above. The analysis of force curves revealed the strengths and locations of molecular interactions established within Rh. 1 (d) Secondary structure of rhodopsin mapped with stable structural segments observed by SMFS and Zn²⁺ binding sites. Arrows locate the starting and end of each structural segment. Numbers in parentheses are the amino acid values obtained from WLC fitting and the numbers without parentheses are the corresponding residue numbers in the rhodopsin sequence. The numbers behind the Cys110-Cys187 disulfide bond are not consistent, since the loop enclosed by the disulfide bridge does not unfold in SMFS.

FIGS. 2 A, B and C show the results for the individual segments determined above of the measurement of force-distance curves of rhodopsin in a buffer solution containing EDTA, or 10, 25, 50, 200 and 400 μM zinc (Zn²⁺). In FIGS. 2 D, E, and F the amino acids of rhodopsin corresponding to the respective beginning of a new unfolding barrier (distinct segment) are marked in a schematic diagram of the secondary structure of the corresponding relevant parts of rhodopsin.

FIG. 3 A is a bar graph of the difference in forces relative to 0 μM Zn²⁺ of unfolding measurements on rhodopsin measured in a buffer solution containing 200 μM Zn²⁺ (black bars) as well as in buffer solutions containing 200 μM Ca²⁺, 200 μM Co²⁺ and 200 μM Cd²⁺. It can be seen that 200 μM Zn²⁺ stabilizes most segments of the receptor whereas the other metals destabilize or have no effect on the majority of the segments. FIG. 3 B shows that 25 mM Mg²⁺ has almost no effect on the stability of rhodopsin.

FIG. 4 shows the ratio of intact Cys110-Cys187 disulfide bonds to broken Cys110-Cys187 disulfide bonds, as well as the probability of broken bonds in rhodopsin when measured in buffer solution containing 1 mM EDTA or increasing amounts of Zn²⁺. Zn²⁺ clearly stabilizes the disulfide bond in a concentration dependent manner.

FIG. 5 shows force-distance-curves of rhodopsin in absence of the stabilizing Cys110-Cys187 bond. 5 (a) A representative force-distance curve reflecting the unfolding of a single rhodopsin molecule lacking the disulfide bond. In contrast to functional rhodopsin having the disulfide bond (FIG. 1), the force curve now exhibits a length of ≈95 nm which corresponds to the length of an entirely unfolded rhodopsin in absence of the Cys110-Cys187 disulfide bond. Fitting each force peak with the WLC model reveals the number of amino acid residues at which the molecular interactions occurred. The values obtained from the WLC model are shown at the end of each fit. 5 (b) A selection of force-distance curves recorded under identical experimental conditions. A comparison of the force curves reveals that some force peaks occur in the majority of curves (main peaks) and some occur in only a small fraction of curves (minor peaks). Each characteristic force pattern of a curve represents molecular interactions that had to be overcome to unfold the protein. Force curves exhibiting the same major and minor force peaks indicate that the rhodopsin molecule unfolded taking the same pathway. 5 (c) The superimposition of several force-distance curves (n=32) enhanced common features among the curves. Major (black) and minor peaks (grey shaded) were fitted using the WLC model as described above. The analysis of force curves reveals the strengths and locations of molecular interactions established within rhodopsin.

### DETAILED DESCRIPTION OF THE PREFERRED EMBODIMENTS

In first broad aspect the invention relates to a method for testing a chemical entity for its capability to modulate a (poly)peptide that is malfunctioning by means of an interaction of said chemical entity and said (poly)peptide, said method using single-molecule force spectroscopy.

One aspect of the invention relates to a method for testing a chemical entity for its capability to modulate a malfunctioning (poly)peptide via an interaction between the chemical entity and the (poly)peptide by means of single-molecule force spectroscopy. Single-molecule force spectroscopy furnishes a force spectrum which is obtained by the steps of:
(a) immobilizing the said malfunctioning (poly)peptide on a support;
(b) attaching the said (poly)peptide immobilized on the said support to a force measuring device;
(c) applying a pulling force to the said (poly)peptide attached to the said force measuring device and measuring the force required for stretching and/or unfolding the said (poly)peptide vs. the elongation of the said (poly)peptide immobilized on the said support and attached to the said force measuring device.

In the method of the present invention the measurements are carried out (i) prior to and (ii) after treatment of the malfunctioning (poly)peptide with the chemical entity. The force spectra so obtained may then be compared. If there is no difference in the spectra, the chemical entity does not modulate the (poly)peptide. If there is a difference, the force spectrum of the corresponding correctly functioning native (poly)peptide untreated with the chemical may be available from the literature, or otherwise may be measured for a further comparison. If the spectrum obtained after treatment of the malfunctioning (poly)peptide is the same as the spectrum of the corresponding correctly functioning native (poly)peptide or if this spectrum shows features that are distinct from the features of the spectrum of the untreated malfunctioning (poly)peptide and resemble the features of the correctly functioning (poly)peptide, the chemical entity modulates the (poly)peptide towards a state of correct functioning, i.e. it restores the correct function of the malfunctioning (poly)peptide partly or entirely. If the spectrum obtained after treatment of the malfunctioning (poly)peptide shows an even greater difference to the spectrum of the correctly functioning (poly)peptide and the malfunctioning (poly)peptide, it may be concluded that the chemical entity tested by the method of the invention does not modulate the (poly)peptide towards a state of correct functioning and may even be toxic for the protein. Sometimes it can be concluded form such a spectrum that the chemical entity stabilizing the (poly)peptide to more than its native state, which is not necessarily disadvantageous.

A “chemical entity” herein means a gaseous, liquid or solid organic, metallo-organic or inorganic compound, including polymers, nucleic acids, peptides, polypeptides and proteins, as it is generally understood by the person skilled in the art, and further includes salts or electrolytes as well as simple or complex ions, e.g. protons and hydroxyl ions (i.e. pH), metal ions, halide ions, sulfate ions, acetate ions, teraalkylammonium ions etc.

In one embodiment of the present invention, the interaction does not involve the formation or disruption of a covalent chemical bond, e.g. the chemical entity does not form a covalent bond within the (poly)peptide and/or does not form a covalent bond with the (poly)peptide and/or does not disrupt a covalent bond in the (poly)peptide.

The chemical entity may, for e.g., be selected from a protein specific ligand such as a protein or a hormone, a synthetic compound such as synthetic acetylcholine, nicotine, glutamate, dopamine, hydroxytryptamine, serotonin, pheromones and interleukin or substitutions thereof, a pharmaceutical compound (e.g. hormone, toxin), a biochemical or biological compound (e.g. acetylcholine, nicotine, glutamate, dopamine, hydroxytryptamine, serotonin, pheromones, interleukin), lipid, (poly)peptide, and/or an inorganic ion, such as Zn²⁺, SeO₃²⁻ and Li⁺.

Preferred chemical entities are organic chemical entities that might function as drugs, in particular small organic molecules that can bind to receptors.

In a preferred embodiment of the present invention, more than one compound suspected of being able to modulate a (poly)peptide is tested. More preferably, the said compound is part of a compound library, and the method of the invention may be used for screening the said compound library for identifying a lead compound for drug development.

In an even more preferred embodiment of the present invention, the screening is a high-throughput screening carried out by multiple force spectroscopes, and/or by multiple force measuring devices (e.g. cantilevers), or by fast-speed force spectroscopy.

A “(poly)peptide” herein is to be understood as comprising synthetic or natural peptides, polypeptides and proteins. Sometimes peptides are characterized as having up to about 30 amino acid residues, polypeptides as having between about 30 and 50 amino acid residues, and proteins as having more than 50 amino acid residues. However, there is no strict delimitation between peptide and polypeptide and between polypeptide and protein.

In a preferred embodiment of the first aspect of the present invention the (poly)peptide is a protein. In a more preferred embodiment of this aspect, the protein is selected from (a) a group consisting of ligand-gated receptors such as nicotinic acetylcholine receptor, hydroxytryptamine serotonin receptor, GABA A receptor, GABA B receptor; (b) G-protein coupled receptors (GPCRs) such as rhodopsin, vasopressin V2 receptor, metabotropic glutamate receptor, interleukin-8 receptor, adrenergic receptor, neuropeptide Y receptor, dopamine 1B receptor, glycoprotein hormone receptor, melanocortin receptor, adenosine receptor, pheromone A receptor; (c) ion channels such as potassium channel, sodium channel, calcium channel, slow voltage-gated potassium channel, NMDA receptor, P2X5 purinoceptor, chloride channel CLC, influenza virus matrix protein M2, cCalsequestrin, arsenical pump membrane protein, MscS and MscL mechanosensitive ion channels, voltage-dependent or gated calcium channel; (d) water channels such as aquaporin 1-10, MIR, GipF; (e) pores such as channel forming colicins, ATP P2X receptor, delta-endotoxin CytB, P2X purinoceptors, mammalian defensin, proteinase inhibitor 117, haemolysin E; (e) antiporters such as sodium/proton antiporter, betaine/proton antiporter, cadmium-transporting ATPase, anion exchange protein, sodium/calcium exchanger, calcium/proton exchanger, sodium/hydrogen exchanger, multicomponent K⁺:H⁺ antiporter, K⁺-dependent Na⁺/Ca⁺ exchanger, multicomponent Na⁺:H⁺ antiporter, sodium/hydrogen exchanger; (f) communication channels such as Gap junctions, connexins; (g) symporters such as LacY, sodium:alanine symporter, Na⁺ dependent nucleoside transporter, glycine neurotransmitter transporter, sodium/glutamate symporter, pentulose kinase; (h) porins such as OmpF porin, maltoporin; (i) ion-gated channels, electrolyte-gated channels and pores, ion pumps, glutamate-gated ion channels, ATP-gated channels; j) mechanosensitive channels such as MscS and MscL; (k) ATP synthases such as F-, V-, or H-type and (I) various enzymes.

Among these proteins, receptors and specifically GPCRs are particularly preferred.

A “correctly functioning (poly)peptide” is a (poly)peptide that fulfils its normal physiological role in a biological individual who has no diseases that can be correlated with that specific (poly)peptide in question.

A “malfunctioning” (poly)peptide is a polypeptide that does not partially or fully fulfil its normal physiological role. The malfunction, for e.g., may be due to a conformational aberration in an environment such as a membrane, due to misfolding, due to incorrect formation of disulfide bridges or due to mutations, often point mutations.

“To modulate a (poly)peptide . . . via an interaction of the said chemical entity and the said (poly)peptide” herein means to change the structure, e.g. the conformation, of a (poly)peptide without altering its amino acid sequence and/or to modulate its functional state by the effect of the chemical entity on the (poly)peptide. “Modulating the malfunctioning polypeptide towards a state of correct functioning” means to modulate the (poly)peptide in such a way that its functioning after the interaction of the chemical entity and the (poly)peptide is shifted towards a more correct or even fully correct functioning of the (poly)peptide compared to its functioning before the interaction.

Preferably, the interaction between the chemical entity and the (poly)peptide does not involve formation or disruption of a covalent bond.

The treatment of the (poly)peptide with the chemical entity to allow this interaction can take place in the fluid cell of e.g. a force microscope e.g. after measuring the untreated malfunctioning (poly)peptide, or it can be carried out separately. The treatment in the fluid cell is suitable when the effect of a chemical entity on a malfunctioning receptor in its natural membrane environment is to be measured. The chemical entity is then simply added to the buffer solution in the fluid cell. In contrast, when a protein, e.g. a receptor, that normally would be misfolded is still present in the endoplasmic reticulum in the cell, a chaperone molecule, in the case of a receptor normally an agonist or antagonist, that can modulate the protein, in this case its folding property, will be added to the endoplasmic reticulum. The treated protein may then be extracted from the cell by means known to the person skilled in the art and studied with single-molecule force spectroscopy according to the invention. Alternatively, if the treated molecule has been incorporated into the membrane due to a more proper folding, it can be unfolded by single-molecule force spectroscopy in its membrane environment.

“Immobilizing a (poly)peptide on a support” can be achieved by a wide variety of means.

The (poly)peptide may be immobilized on the support in an isolated form that may, for e.g. be a three-dimensional or two-dimensional crystalline form, an amorphous form, a dimeric form, a trimeric form. The (poly)peptide may also be present in a biological or artificial environment, such as incorporated into a membrane, embedded in a lipid bilayer (biological or synthetic), attached to a membrane, in a vesicle, cell or on any biological interfaces, such as cell membranes, membranes of vesicles, lipid membranes, extracellular matrix and compounds thereof; in or attached to other biological structures, such as lipids, collagen, actin, microtubules, cytoskeleton, peptides, protein and protein complexes, antigens, antibodies, aggregates such as amyloid fibres and plaques, bone, nucleic acid molecules, fibrils, fibres and tissues in general and attached to small biological or synthetic molecules, such as amines, antibiotics, hormones, enigmols, supramolecular cylinders [Fe₂L₃]⁴⁺. In some types of force spectroscopy this biological or artificial environment may directly serve as a carrier.

Synthetic lipid bilayers embedding proteins can be generated from their constituents in vitro. To this end, lipidic compounds such as phospholipids, sphingolipids, glycolipids, cholesterol and other biological or synthetic lipidic compounds are mixed in the presence or after addition of the (poly)peptide.

The support may be selected from inorganic solid state materials, e.g. mica, graphite, silicon, gold, gallium arsenide; polymers, e.g. polyethylene, polypropylene, polystyrene, acrylate, collagen; synthetic membranes, e.g. block-copolymers or membranes assembled from synthetic lipids; synthetic scaffolds, e.g. peptide hydrogels, hydrogels, polymer nanofibres, peg-pamam sar polymers, wherein the aforementioned materials may be present as flat carriers or in the form of beads; and as mentioned above, in some types of force spectroscopy, the above-mentioned biological or artificial environment of the (poly)peptide.

The immobilization of the (poly)peptide in its isolated form or further association with one of the above mentioned biological or synthetic structures can for example be achieved by covalently binding the (poly)peptide to the support, by non-covalently binding the (poly)peptide to the support or by adsorbing the (poly)peptide on the support. The covalent binding can for example be achieved e.g. by disulfide bonds. Non-covalent binding can be achieved by non-covalent interactions that may, for e.g. be mediated by antibodies or other biological molecules such as avidin, biotin, histidine tags, collagen binding domains, streptavidin, fibronectin, which are capable of specifically attaching the (poly)peptide. Adsorption may be based on hydrophobic, hydrophilic, electrostatic, polar or ionic interactions between the (poly)peptide and the support.

Particularly preferred herein are (poly)peptides that are present in their natural membrane environment and are attached to an inorganic support, preferably mica, by means of adsorption.

It should be mentioned here that the immobilization procedure is performed under aqueous conditions and the subsequent measurements performed in a fluid cell containing a suitable buffer solution, since most (poly)peptides would be denatured in air.

The term “force spectroscope” is to be understood in its broadest meaning and includes any apparatus by which a small force can be measured. This includes atomic force spectroscopes (microscopes), force spectroscopes that employ magnetic or optical tweezers, scanning probe spectroscopes (microscopes) and others. Particularly preferred herein is an atomic force spectroscope (microscope).

Consequently, the term “force measuring device” of a force spectroscope includes devices such as a cantilever of an atomic force spectroscope; magnetic tweezers; optical tweezers; a protein or protein complex that may be attached to beads or the tip of a cantilever of an atomic force microscope; any other biological molecule or synthetic molecule as defined above with respect to the environment in which a protein may be present that may be attached to, for e.g. beads, the tip of a cantilever and the like; the surface of a force apparatus such as mica, graphite, silicon, gold, gallium, arsenide; a membrane, e.g. a cell membrane, vesicle, lipid bilayer, bloc-copolymer; and the probe of a scanning probe microscope or a scanning probe spectroscope. Particularly preferred herein as a force measuring device is the cantilever of an atomic force microscope.

The (poly)peptide may be attached to the measuring device by covalent or adsorption means. The latter are based on hydrophobic, hydrophilic or steric interactions and/or interactions by charge (e.g. van der Waals, ionic, polar, electrostatic interactions). For attaching the (poly)peptide to the measuring device, the measuring device and the (poly)peptide are brought into contact and a force of usually about 300 to 1500 pN is applied which may result in one terminal end of the (poly)peptide being bound to the measuring device either via a covalent bond or via enforced non-specific adsorption. (It should be mentioned that, e.g. in the case of a tip of a cantilever of an atomic force microscope not in all trials the terminal end of the (poly)peptide will be bound, but rather another part of the (poly)peptide.) Particularly preferred herein is to attach the (poly)peptide to the tip of a cantilever by means of adsorption.

The pulling force applied to the (poly)peptide is generally in the range of about 1 to 500 pN. By applying a strong enough pulling force in this range the (poly)peptide is stretched and/or unfolded. When a pulling force is applied that is strong enough to unfold the protein the force applied plays the role of a denaturant leading from continuous extension of the protein to complete unfolding of its three-dimensional structure. The force required for withdrawing the measuring device attached to the (poly)peptide from the carrier or support (this is very confusing throughout the text. Carrier is also used for membranes and also for supports) represents the pulling force. As soon as this pulling force exceeds the stability of the protein, it induces the sequential unfolding of its three-dimensional structure.

In a preferred embodiment of the present invention, the pulling force is exerted by the force measuring device. In the case of a cantilever it is moved to stretch the (poly)peptide.

In a further preferred embodiment of the present invention, the pulling force is exerted by the support. In this case the support is moved to stretch the protein.

In another embodiment of the present invention, the pulling force is exerted by the (poly)peptide. In this case the (poly)peptide exerts forces that move the force measuring device, e.g. the cantilever.

In a particularly preferred embodiment of the present invention, the pulling force is exerted by a mixture of two or three of the above embodiments.

The unfolding of a protein usually takes place in segments. Each segment has its own stability barrier which must be overcome by the pulling force. Once the barrier is overcome, the segment in question unfolds spontaneously. When the pulling force applied to the protein is measured vs. the elongation thereof (or vs. the distance of the measuring device to the sample), one obtains the so-called force-distance curve, also called force spectrum, which shows a typical sawtooth curve if the protein unfolds in segments. If the sequence of the protein is known, the beginning of each segment can be assigned to a certain amino acid or at least group of amino acids, since the elongation is also measured and the length of the unraveled chain can be assigned to the number of amino acids by the wormlike chain (WLC) model.

When a malfunctioning protein is unfolded, its force spectrum will be different from the force spectrum of the corresponding correctly functioning protein. The reason is that the three-dimensional organization of the protein is different in a malfunctioning protein. This may be an altered conformation due to an altered environment (e.g. lipid environment, disturbed dimer (homodimer or heterodimer), or trimer association), due to “wrong” disulfide bridges and/or due to a mutation (usually point mutation). In more dramatic cases, the protein is fully misfolded, and if it is a membrane protein it may not be able to occupy its place in or on the membrane.

An aberrant conformation can be detected in the force spectrum of a protein. Usually it leads to destabilization of the protein or a segment of the protein which can be detected because the unfolding of the protein or the segment of the protein requires less force when destabilized. In more extreme cases the onset of a segment as described above will be shifted or disappear or a new segment will appear. By means of the wormlike chain model it can be determined which amino acid(s) take(s) part in the destabilization.

When disulfide bridges in a malfunctioning protein are cleaved or appear in another location, the length of the unfolded protein changes, since disulfide bridges remain intact in force spectroscopy and e.g. a loop formed by a disulfide bridge will not be unraveled.

Point mutations can cause conformational changes, altered disulfide bridges and complete misfolding. A complete misfolding will lead to a greatly altered force spectrum.

A chemical entity may act on various parts of the protein molecule. It may lead to a new covalent bond within the protein and/or from a covalent bond with the protein and/or disrupt a covalent bond within the protein. Often this would be a toxic chemical entity. More preferred is the case wherein the chemical entity forms a non-covalent bond with the protein. This may be e.g. mainly a steric interaction combined with hydrogen bonding, such as an antigen/antibody interaction or a ligand/receptor interaction, or a charge interaction such as the binding of a cation to negatively charged groups or negatively polarized groups on the protein, and the like.

The following discussion uses receptors as a non-limiting example. It should be noted, however, that similar considerations apply to other proteins as well.

When a ligand binds to a receptor (which would be a malfunctioning receptor in the first aspect of the present invention), the interaction will bring about a change in the conformation and the stability of the binding pocket of the receptor. This change will be marked and will be detected by a substantial alteration of the force spectrum, which in most cases will reveal a stabilization of the binding site (increase in pulling force). Since the ligand necessarily binds to a part of the receptor exposed to the extracellular space, the exact situation of the binding site can be elucidated by means of single-molecule force spectroscopy, the wormlike chain model, the known amino acid sequence of the receptor and perhaps the knowledge of at least some structural details of the receptor.

Other changes in the spectrum may also appear. The binding of a ligand may be accompanied by other conformational rearrangements within the three-dimensional organization of the protein affecting isolated regions within the secondary structure of the protein such as an entire alpha helix or beta sheet arrangement, or affecting entire subunits of a protein. Again, the conformational rearrangements have an impact on the intrinsic stability of the receptor, even if only a few amino acids are affected. This change in stability can again be measured by the method of the present invention and assigned to specific parts of the protein using the wormlike chain model. Therefore, not only a stabilization or destabilization of the receptor may be detected, but also the site of this stabilization or destabilization within the protein.

In the case of malfunctioning receptors, the influence of allosteric ligands is of particular interest. These ligands can change the conformation of the receptor without occupying the binding site of the receptor's native ligand. Thus, the receptor may be modulated by the allosteric ligand to become more sensitive or less sensitive or not sensitive at all for its native ligand. Again, this is associated with a change in conformation at least at the binding site of the allosteric receptor, but often also at the binding site of the native receptor and at further sites within the protein. As mentioned above, these changes as well as their locations within the molecule can be measured and determined by force spectroscopy.

If in the case of a malfunctioning receptor the receptor has been treated with a potential ligand, the comparison of the force spectrum of the treated receptor with a force spectrum of the untreated malfunctioning receptor and the untreated correctly functioning receptor will reveal the nature of the interaction of the potential ligand with the receptor, i.e. if it is able to restore the function of the malfunctioning receptor partially or fully. The closer the force spectrum of the malfunctioning receptor treated with the ligand will be to that of the untreated correctly functioning receptor, the better a potential medicament the ligand will be. “Untreated correctly functioning receptor” in this context means that the receptor has not been treated with the ligand to be tested. It does, however, not exclude that the force spectrum of the correctly functioning receptor has been acquired in the presence of the native ligand of the receptor.

By this comparison it may be elucidated in which part(s) of the receptor the malfunction resides.

In the case of toxic ligands it is to be expected that the receptor becomes either destabilized or so strongly stabilized that it is not able to change its conformation anymore for transmitting a signal.

In another broad aspect the invention relates to a method for testing a chemical or physical entity for its capability to interact with a G protein-coupled receptor (GPCR) in its natural membrane environment, said method using single-molecule spectroscopy.

In another aspect the invention relates to a method for testing a chemical or physical entity for its capability to interact with a G protein-coupled receptor (GPCR) in its natural membrane environment, the method comprising the steps of
(a) immobilizing said GPCR in its natural membrane environment on a carrier;
(b) attaching said immobilized GPCR on said carrier to a force measuring device of a force spectroscope;
(c) applying a pulling force to said GPCR attached to said force measuring device and measuring the force required for stretching and/or unfolding said GPCR vs. the elongation of said GPCR immobilized on said carrier and attached to said force measuring device, wherein the measurements are carried out (i) prior to and (ii) after treatment of said GPCR with said chemical or physical entity;
(d) optionally comparing a force spectrum of said GPCR obtained prior to said treatment and a force spectrum of said GPCR obtained after said treatment, and
(e) optionally concluding from a difference of the force spectra of (d) whether said physical or chemical entity interacts with said GPCR.

Preferably, the difference of the force spectra of (d) allows the conclusion whether the interaction stabilizes or destabilizes the said GPCR and where in the GPCR the interaction has occurred.

Preferably, the interaction between the chemical entity and the GPCR does not involve formation or disruption of a covalent bond.

The chemical entities in these aspects are the same as those discussed above for the first aspect of the present invention. The physical entities may be selected from light, voltage or current. Physical entities may elicit formation and breaking of covalent bonds as well as conformational changes.

The natural membrane environment may include just the membrane in which the GPCR is embedded, or further cellular or tissue structures surrounding the membrane.

The other definitions given above in the context with the first aspect of the present invention, which are also appropriate for the second aspect, are to be applied to the second aspect.

The entire disclosure of all references cited in this application is herein incorporated by reference.

The invention will be further explained by the following non-limiting examples that are intended to be illustrative only and should not be construed as limiting the scope of the invention, which is defined in the appended claims. The examples use rhodopsin as a prototype of a protein and more specifically of a GPCR. The person skilled in the art will recognize that the principles explained in the examples can be applied to other (poly)peptides, more specifically proteins and in particular GPCRs.

### EXAMPLES

### Example 1

### Rhodopsin in the Absence and Presence of Zinc

Rhodopsin is the light receptor that initiates phototransduction in the rod outer segments (ROS) of the eye. Rhodopsin is a prototypical GPCR. The specific localization of rhodopsin in the internal discs and its high expression level (constituting >90% of all proteins in disc membranes) have facilitated studies in this system.

In this example force spectra of rhodopsin in native bovine ROS disc membranes were obtained in the presence of EDTA and various concentrations of zinc by means of single-molecule force spectroscopy using the cantilever of an atomic force microscope as the force measuring means and means to measure the extension of the receptor under the pulling force of the cantilever.

It is known that functional rhodopsin binds zinc ions in its active as well as inactive (dark) state. There is; one zinc binding site within the transmembrane helices (Glu122 and His211) that is physiologically of great relevance, since mutations of His211 that cannot bind zinc any more cause the disease retinitis pigmentosa that eventually leads to vision loss.

Moreover, rhodopsin possesses further zinc binding sites outside the transmembrane helices both on the extracellular and cytoplasmic part of the protein. Since the zinc concentration in the retina is possibly as high as in the brain, (estimated to range from about 150 to as much as 300 μM), it is to be expected that zinc also binds to these extracellular and cytoplasmic binding sites under physiological conditions. This would mean that a correctly functioning rhodopsin binds to more than one zinc ion.

Conversely, it can be concluded that a rhodopsin molecule that is not bound to (enough) zinc, when there is not enough zinc available in its environment, is malfunctioning.

It is known that night blindness can be caused by zinc deficiency. It was believed that this was caused by a malfunction of the enzyme in the retina that converts all-trans-retinal into 11-cis-retinal, but it is now known that this enzyme is zinc-independent. Today it may very well be assumed that night blindness is directly caused by a malfunction of rhodopsin itself.

### Zinc Binding Sites

It is known that rhodopsin contains a high affinity zinc-specific coordination site within its 11-cis-retinal chromophore binding pocket [29]. On the basis of mutagenesis experiments of the three amino acids forming the binding site the authors of reference [29] hypothesized that zinc stabilizes the ground (dark) state of rhodopsin. During photoactivation the zinc coordination may then become disrupted leading to structural changes and the formation of photoactivated intermediates. Without the stabilization of the ground state by zinc, the receptor does not seem work properly.

The authors of the above-mentioned reference used inductively coupled plasma mass spectrometry and fluorescence detection of zinc. These methods are not suitable for screening a large number of chemical entities; the latter might be other metal ions, such as copper, cobalt, cadmium etc., but also metallo-organic or organic molecules in the present case. A more cost efficient method for solving the above and similar problems is single-molecule force spectroscopy (SMFS), with at least equally precise results with respect to determining the stability of the receptor and the binding site of chemical entities, if desired.

Recording Force Spectra with and without Zinc (Correctly Functioning and Malfunctioning Receptor)

First, a force spectrum of rhodopsin in a buffer solution containing neither EDTA nor zinc was recorded. The experimental details are given in the Experimental Section.

Since EDTA has been used in the preparation of the disc membranes, essentially, all of the zinc present in the disc membranes except for the zinc buried in the helices of rhodopsin is removed by EDTA. Accordingly, there is no zinc that can bind to the extracellular and cytoplasmic binding sites of rhodopsin. Thus, the spectrum of rhodopsin in such an environment can be considered to correspond to a spectrum of the malfunctioning receptor, since the receptor does nor function properly in the absence of zinc.

Through addition of a suitable chemical entity—in the present case this will be zinc ions, but the addition of an organo-zinc compound that is able to release zinc ions could be contemplated as well—the receptor can be modulated towards correct functioning. It was determined that the stability of rhodopsin increased at zinc concentrations above about 50 μM, and the highest stability of dark state rhodopsin could be attained in the presence of 200 μM zinc. It may be assumed that this corresponds approximately to a healthy physiological zinc concentration in the retina. Therefore, rhodopsin in the presence of this zinc concentration may be assumed to be the correctly functioning receptor or at least close to it.

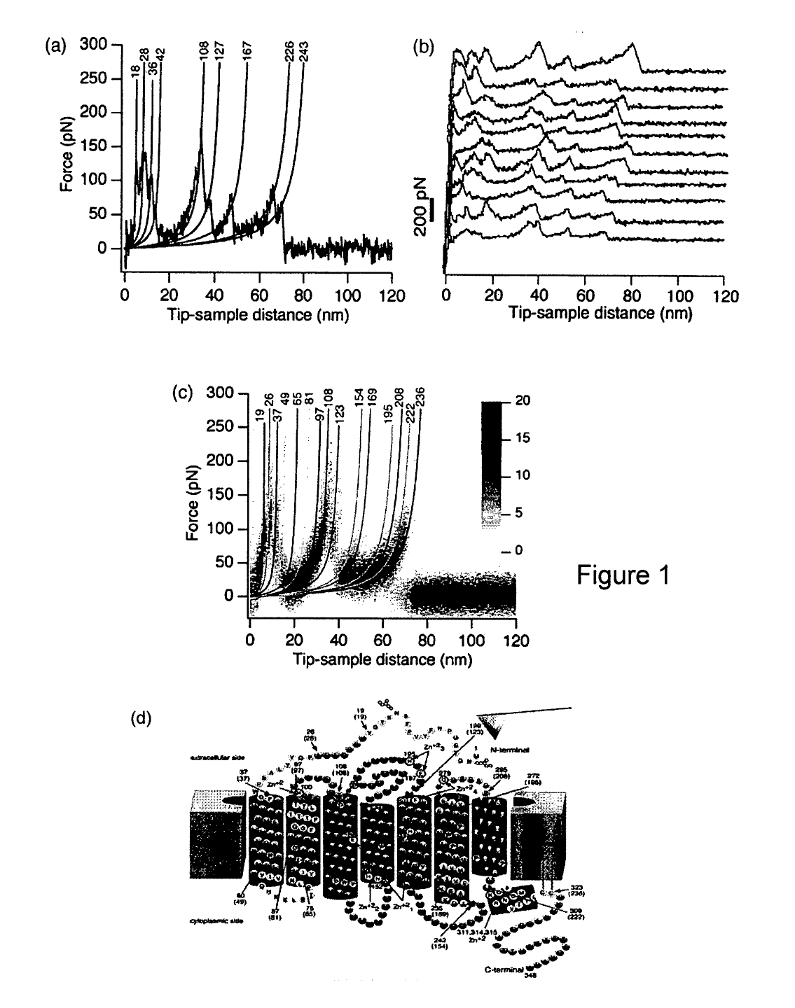

A series of typical force spectra of native rhodopsin having a disulfide bond between Cys110-Cys187 (hereinafter: native rhodopsin) in a buffer solution containing neither EDTA nor zinc is shown in FIG. 1. The effective length of the stretched polypeptide chain in the force-distance curves is ≈65 nm, since the loop formed by the disulfide bridge is not stretched in force spectroscopy (the forces to break the covalent disulfide bond—at least 10 times those needed to unfold the protein—are not detected in the experiment).

The spectra were obtained in the following way. At first native ROS disc membranes containing rhodopsin were imaged in buffer solution by atomic force microscopy (AFM) prior to SMFS studies. A region in the center of the membrane was used for SMFS. A single rhodopsin molecule was attached to the AFM stylus (tip of the cantilever) by contacting it with the surface of a single-layered disc membrane. A rhodopsin peptide loop or terminal end adsorbed non-specifically to the stylus upon applying a contact force of ≈1 nN for about 0.5 to 1 s. A single rhodopsin molecule adsorbs to the AFM stylus through its N-terminal region thus establishing a molecular bridge, which can then be used to exert a mechanical pulling force to unfold the protein.

### The Force Spectra Obtained—Stabilization of Rhodopsin by 200 μM Zinc

The force-distance curves obtained in the course of this pulling are shown in FIGS. 1 (a) and (b). Every single force peak in these spectra, corresponding to a segment of the protein that unfolds with a distinct force barrier, was fitted using the wormlike chain model which describes the stretching of a polymer (Reif. M., et al. (1977), Science 276, 1109-12). This procedure revealed the number of amino acid residues (aa) stretched for the particular force peak (segment) in question, and allowed to locate molecular interactions established by the protein. Individual force-distance curves showed variations between each other (see FIG. 1 (b)). Statistically significant insights were gained by performing the same experiment several hundred times. FIG. 1 (c) shows the numbers of the amino acids where the onset of a new unfolding barrier is located. FIG. 1 (d) shows a schematic model of rhodopsin in its disc membrane wherein the zinc binding sites are indicated (one in the transmembrane helices, two on the extracellular side and one on the cytoplasmic side).

Several secondary structural segments corresponding to the individual force peaks could be identified and their mechanical stabilities could be characterized. The force required to unfold a secondary structure segment can be taken as a measure of its stability. An increase or decrease in the unfolding force of such a segment under different conditions would imply a change in its stability.

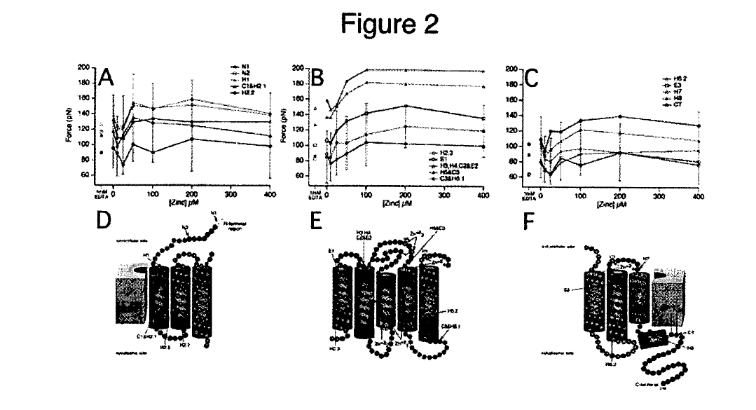

In the same way the force spectra of rhodopsin in a buffer solution containing EDTA, or 10, 25, 50, 200 and 400 μM zinc were obtained. The results for the individual segment determined above in the buffer containing EDTA or zinc are shown in FIGS. 2 A, B and C. The amino acids of rhodopsin corresponding to the respective onset of a new unfolding barrier (distinct segment) are marked in the schematic diagram of the corresponding relevant parts of rhodopsin in FIGS. 2 D, E, and F.

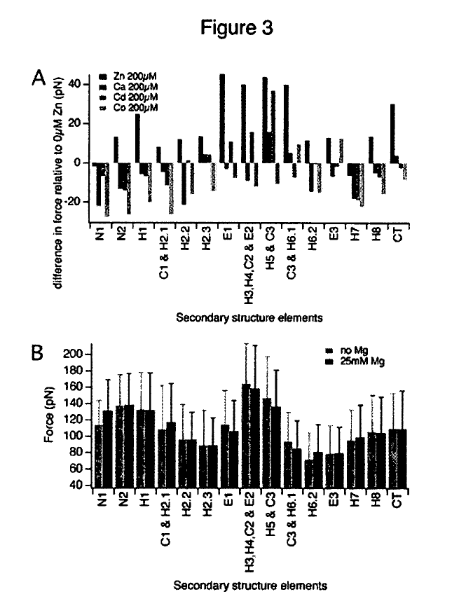

FIG. 3 A is a bar graph of the difference in force relative to 0 μM zinc when rhodopsin is measured in a buffer solution containing 200 μM zinc (black bars) as well as in buffer solutions containing 200 μM calcium, 200 μM cobalt and 200 μM cadmium. It can be seen that 200 μM Zn²⁺ stabilizes most segments of the receptor whereas the other metals destabilize the majority of the segments.

FIG. 3 B shows that 25 mM magnesium has almost no effect on the stability of rhodopsin.

The extracellular domain which forms a sort of lid on the rhodopsin molecule hosts two zinc binding sites containing histidine residues. It seems that the stabilization of this “lid” generally stabilizes also most of the helix segments (H1 to H8 in FIG. 3 A). Zinc therefore seems to exert a global effect on the stability of rhodopsin.

It should be mentioned here that in this experiment the zinc binding sites were not particularly monitored. In principle, however, it is possible to detect the localisation of a binding site of a chemical entity in a protein by SMFS, as was shown by our earlier patent application cited above. The method consists in monitoring the rise of (a) new peak(s) or disappearance of peaks in that part of the force spectrum where the binding site is suspected when the chemical entity is added to the protein.

### Unfolding Probability of Secondary Structure Elements

Each unfolding event has a certain probability of occurring. The superimposition of many force-distance curves enhances unfolding events occurring repeatedly (FIG. 1 (c)) while rarely occurring single events are masked by noise.

Force peaks in the superimposition in the present case were fitted using the wormlike chain model, which correlated with the averages obtained from fitting each peak in individual curves (FIG. 1). Results of the wormlike chain model analyses were mapped onto the secondary structure of rhodopsin. Thus, structural segments could be determined that establish molecular interactions strong enough to be detected in the experiments. The characteristic sequence of the force peaks in an unfolding spectrum represents a unique unfolding pathway of that particular rhodopsin molecule. Statistical analyses allow calculations of the frequency (probability) with which the rhodopsin molecules selected a particular intermediate in the unfolding pathway.

Unfolding native rhodopsin by SMFS in the absence and presence of 10, 25, 50, 100, 200 and 400 μM zinc⁺ leads to a change in the occurrence probabilities of the unfolding pathways. In contrast, 200 μM of other metal ions did not drastically change the unfolding pathways. We take a change in the probabilities of unfolding pathways as a measure of changing molecular interactions in the rhodopsin molecule upon zinc binding.

To summarize, native rhodopsin without zinc being bound to its extracellular and cytoplasmic binding sites shows altered molecular interactions that impair the functioning of the receptor. The receptor may be called malfunctioning. It seems that the addition of the chemical entity zinc in a concentration of around 200 μM restores the correct functioning of the receptor. In this case the spectrum of the malfunctioning receptor treated with the chemical entity and the spectrum of the correctly functioning native non-treated receptor (if measured in its natural environment particularly with respect to the zinc concentration) would probably be identical.

### Example 2

### Absence of Cys110-Cys187 Disulfide Bond in Rhodopsin

In our unfolding experiments of rhodopsin in ROS disc membranes, we surprisingly observed another form of rhodopsin than that having the Cys110-Cys187 disulfide bond intact, namely a form having a disrupted Cys110-Cys187 disulfide bond and perhaps instead a Cys185-Cys187 bond. This altered disulfide bond has been known in mutants of rhodopsin that lead to the disease retinitis pigmentosa. In the present case, however, no mutations seem to be involved.

In contrast to the native form of rhodopsin having the Cys110-Cys187 bond and a length of ≈65 nm, the form lacking the Cys110-Cys187 bond exhibits a length of ≈95 nm. This is due to the fact that the loop not unraveled by SMFS in the Cys110-Cys187 form is unfolded in the 95 nm form.

The 95 nm form of rhodopsin is not a functional receptor, since the Cys110-Cys187 bond is critical for the stability and function of the receptor.

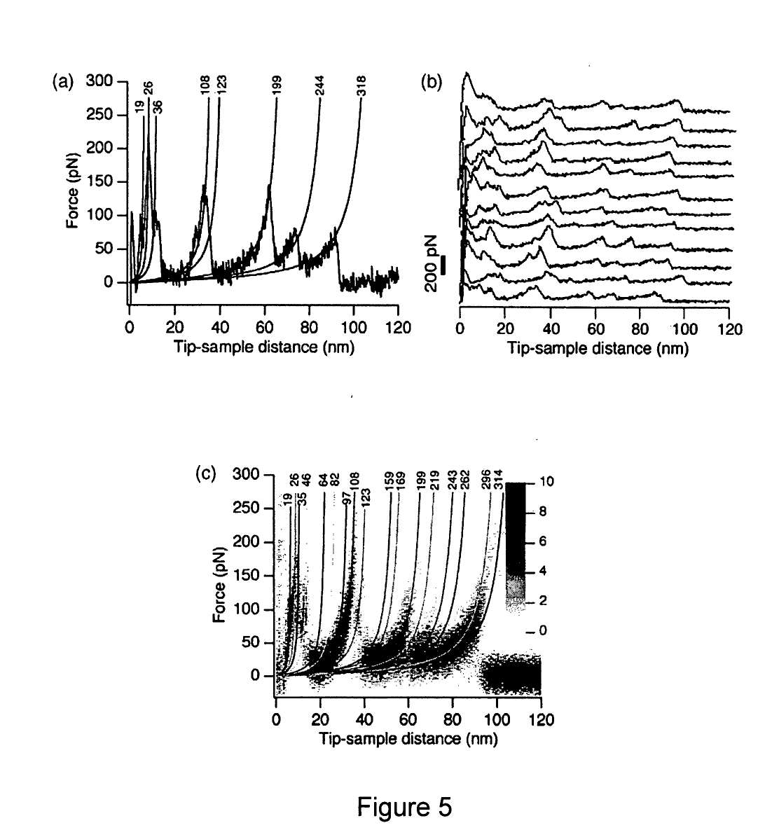

The force spectrum of the dark state of 95 nm rhodopsin is shown in FIG. 5 (a). A selection of several force spectra is shown in FIG. 5 (b). FIG. 5 (c) shows the superimposition of 32 force spectra as described above with reference to the Cys110-Cys187 force spectrum.

These force spectra were obtained in substantial absence of zinc, since EDTA was used in the preparation of the ROS disc membranes. With these preparation conditions the ratio of Cys110-Cys178 rhodopsin and 95 nm rhodopsin in the ROS disc membranes was found to be about 2:1 when the force spectra were measured in a buffer solution not containing any chemical entity that binds to rhodopsin.

A comparison between the spectra of Cys110-Cys187 rhodopsin and 95 nm rhodopsin reveals that the force peaks at a tip-sample distance of <40 nm of the latter essentially remain at the position observed for Cys110-Cys187 rhodopsin while they are clearly different at higher tip-sample distances. In absence of the Cys110-Cys187 bond the structural segment held together by the S—S bond in Cys110-Cys187 rhodopsin could now unfold in individual steps. Some segments of the receptor were stabilized by significantly different amino acid regions while others were not influenced.

The ratio of the Cys110-Cys187 rhodopsin force-distance curves and the 95 nm rhodopsin force-distance curves was changed in the presence of dithiothreitol and N-ethylmaleimide. The ratio of the 65 nm curves to the 95 nm curves was 1:2 in the presence of dithiothreitol and 7:1 in the presence of N-ethylmaleimide. Dithiothreitol likely disrupts the Cys110-Cys187 disulfide bond, and N-ethylmaleimide alkylates Cys 185. The action of these two compounds likely occurs during the unfolding of rhodopsin, since the residues modified by these reagents are not accessible in the native structure of the protein, as can be judged from the known crystal structure of rhodopsin.

There are at least two explanations that can account for force spectra having lengths corresponding to the unfolded rhodopsin molecules with an absent Cys110-Cys187 bond. The first possibility is that a population of rhodopsin molecules exists in the disc membranes that do not contain the S—S linkage. The stoichiometry of the rhodopsin molecules with and without the native S—S bond would be 2:1 in the disc membranes. An alternative explanation involves the replacement of the native S—S bond between Cys110 and Cys187 by a non-native bond linking Cys185 and Cys187. This has been observed under certain conditions [47, 48, 49], and therefore the unfolding of the polypeptide chain in some instances may promote the non-native S—S bridge between the adjacent Cys residues. This latter explanation is supported by the effect of N-ethylmaleimide on the unfolding of rhodopsin, where a 3.5-fold increase in the proportion of the 65 nm curves is observed. Since N-ethylmaleimide alkylates Cys 185, no Cys185-Cs187 disulfide linkage can be formed. However, even in this case there still remain ⅛ of the total rhodopsin molecules that lack the Cys110-Cys187 bond. These molecules might exist in the disc membrane.

There is an effect of added zinc ions on the ratio of the Cys110-Cys187 rhodopsin and the 95 nm rhodopsin.

A putative zinc binding site has been proposed to involve His211 [29]. His211Pro is a naturally occurring point mutation that causes misfolding and results in retinitis pigmentosa. Misfolding of this and other point mutations results in the replacement of the native Cys110-Cys187 stabilizing disulfide bond with the non-native Cys186-Cys187 destabilizing disulfide bond [32]. The disruption of the zinc binding site involving His211 has been proposed to underlie the mechanism of those effects [29].

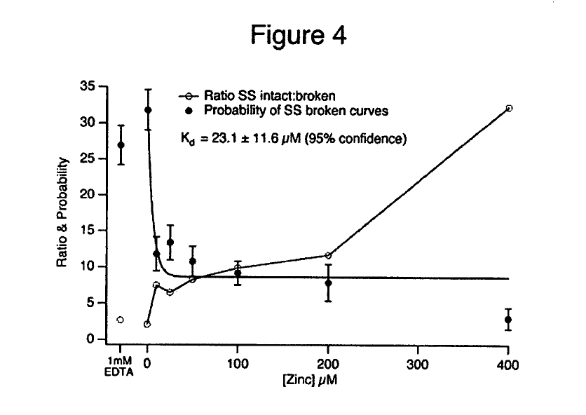

The effect of zinc on the ratio of force-distance curves corresponding to the pulling of 65 nm rhodopsin in presence of the native disulfide bond to the pulling of 95 nm rhodopsin in absence of the native disulfide bond is consistent with this proposal. Increasing zinc concentrations favour the level of 65 nm rhodopsin (see FIG. 4), and therefore the apparent stabilizing effect of zinc is reflected in the maintenance of the native Cys110-Cys187 disulfide bond. This points to a physiological role of zinc in the stabilization and promotion of the proper folding of the receptor molecule.

A possible drug for preventing the misfolding of mutant rhodopsin could therefore be a chaperone molecule that stabilizes the mutant rhodopsin at those lost binding sites of zinc, where a binding of zinc ions is not possible any more due to a mutation during the folding in the endoplasmic reticulum and later on in the membrane.

### Experimental Section

### ROS Disc Membrane Preparation

All experimental procedures were carried out under dim red light. Centrifugation Steps were performed at 4° C. ROS were purified from fresh bovine retinas as described [33] and stored at −80° C. To obtain disc membranes, ROS membranes were resuspended using a glass hand-held homogenizer in 13 mL of buffer A (2 mM Tris-HCl, pH 7.4) and incubated overnight at 4° C. The membrane suspension was centrifuged at 213,500×g for 30 min. The membranes were washed twice with 13 mL of buffer A and three times with 3 mL of buffer B (2 mM Tris-HCl, 150 mM NaCl, 2 mM EDTA, pH 7.4). Membranes were collected each time by centrifugation at 26,500×g for 30 min. The membranes were resuspended in buffer A and used for SMFS and AFM imaging. Alternatively, membranes were resuspended in buffer C (67 mM potassium phosphate, 1 mM magnesium acetate, 0.1 mM EDTA, 1 mM DTT (dithiothreitol), 18% sucrose, pH 7.0) and stored at −80° C. Membranes stored in buffer C were washed twice with buffer A prior to SMFS studies.

For cleavage experiments, ROS membranes (2 mg/mL) resuspended in Ringer's buffer (10 mM Hepes, 130 mM NaCl, 3.6 mM KCl, 2.4 mM MgCl₂, 1.2 mM CaCl₂, 0.02 mM EDTA, pH 7.4) were treated with endoproteinase Glu-C (Roche Applied Science, Indianapolis, Ind., USA) at a substrate to enzyme ratio of 10:1 (w/w). Membranes were digested with the enzyme for 2 h at room temperature and 1.5 h on ice. The reaction was terminated by washing membranes three times with Ringer's buffer containing 5 mM benzamidine (1 mL) and one time with buffer A (1 mL). Membranes were resuspended in buffer A for SMFS studies. For solubilization of rhodopsin (Rh), ROS membranes were resuspended in 20 mM BTP, 150 mM NaCl, 1% n-dodecyl-β-D-maltoside, pH 7.4. The membrane suspension was shaken at room temperature for 10 min and then centrifuged to remove insoluble material at 164,000×g for 30 min. Protein concentration was determined using the Bradford Protein Assay Kit from Bio-Rad Laboratories (Hercules, Calif., USA).

### Attachment of Rhodopsin (Rh) to the AFM Cantilever

Rh was attached nonspecifically to a Si3N4 cantilever by applying a contact force of ˜1-1.5 nN between the AFM stylus and the membrane surface. This method has been tested on several membrane proteins and allows a much higher throughput as compared to specific attachment via thiol-gold linkage [34,35,36,37].

### AFM Imaging and SMFS

AFM imaging and force spectroscopy were performed as described elsewhere [34,39]. Disc membranes were adsorbed onto a freshly cleaved mica surface as described previously [38]. For force measurements, the AFM stylus was approached to the Rh surface while applying a constant force of ˜1 nN. After a contact time of ˜0.5-1 s, the stylus was retracted from the membrane surface at a constant velocity of 300 nm/s. All experiments were performed in 150 mM KCl, 25 mM MgCl₂, 20 mM Tris, pH 7.8, at room temperature. In experiments where rhodopsin was treated with 1 mM EDTA (Sigma), ZnCl₂ (Fisher Scientific), CaCl₂, CdCl₂, CoCl₂, or CuCl₂, the buffer was supplemented with the specified amounts of the reagent and the pH adjusted to 7.8. When assayed with 0, 10, 25, 50, 100, 200 and 400 μM ZnCl₂, rhodopsin in native ROS disc membranes was diluted in the dilution buffer with the required amount of ZnCl₂ and incubated on ice for ˜40 mins—1 h in complete darkness. In the case of other metal ions the sample preparation was carried out in a similar way. In the experiment where 1 mM EDTA was used, 25 mM MgCl₂ was not added to the buffer. All dilutions were carried out under dim red light and experiments performed in a completely dark room. Force curves were collected over a period of less than 1 h to prevent bleaching of rhodopsin. The red laser on the AFM promoted minimal bleaching of Rh. ROS membranes solubilized in n-dodecyl-β-D-maltoside were bleached less than 5% after incubation under the AFM laser for 1 hr.

To rule out statistical errors due to cantilever spring constant deviations the SMFS experiments were performed on each Rh sample using ˜20 different cantilevers from the same batch. Also, the experiments were performed using two different AFM equipments, viz., Picoforce (dl-Veeco, USA) and Multimode (dl-Veeco, USA). Force-distance curves obtained from the two instruments were analyzed separately. The unfolding forces measured in the two cases agreed within <20% and those of WLC fits were the same. Spring constants of the 200 μm long silicon nitride AFM cantilevers (NPS, Veeco Metrology; nominal spring constant −0.08 N/m) were calibrated in buffer solution using equipartition theorem [40,41]. All cantilevers exhibited similar spring constants within the uncertainty of the above calibration method (˜10%).

### Selection and Analysis of Force-Extension Curves

In the first step, the force-distance curves were separated based on their lengths. Following this procedure we were able to group the curves into two classes based on length: one class had a length of ≈65 nm and the other a length of ≈95 nm. Of the total curves collected 11% belonged to the ≈65 nm length class and 4% to the ≈95 nm length class (n=670). The length selection helped us to classify the curves from Rh molecules with an intact native S—S bond (Cys110-Cys187, ≈65 nm), and an absent native S—S bond (≈95 nm). Force extension curves exhibiting an overall length of 65 nm (or ≈95 nm) reflect completely unfolded and extended Rh molecules attached with their N-terminal end to the AFM stylus [42,43]. It is important to mention that a good fraction of curves have smaller lengths due to attachment of the AFM stylus to one of the loops or detachment of the molecule from the stylus during unfolding. We discarded these curves during the analyses. All force-distance curves exhibiting similar overall unfolding spectra and lengths were selected and aligned using identical procedures and criteria established previously.²⁴ Every peak of a single force curve was fitted using the wormlike chain (WLC) model with a persistence length of 0.4 nm and a monomer length of 0.36 nm [43]. The number of extended amino acid residues at each peak was then calculated using the contour length obtained from the WLC fits. This allowed for the assignment of unfolding events to structural segments of Rh as described [44]. To measure the unfolding force and probability of unfolding for each individual structural segment, every event of each curve was analyzed.

As mentioned above the molecule could detach from the AFM stylus during unfolding. Therefore, for the cleavage experiments with endoproteinase Glu-C it is important to be certain that the <40 nm length force-distance curves are mainly from the N-terminal fragment of the molecule and not from the detached molecule. To get comparative statistics for the cleavage experiments, we estimated the percentage of curves showing the same molecular unfolding pattern (peaks at amino acids 19, 26, 37 and 97, 108, 123) due to the detachment of the molecule from the total number of curves obtained from the uncleaved native Rh sample, and compared it to the fraction of curves obtained from the enzymatically treated sample. Of the total curves obtained the fraction of short curves (<40 nm) was 23% in the case of untreated sample and 55% for the digested sample, whereas the fraction of ≈65 nm and ≈95 nm long curves decreased to 2% in the enzymatically treated sample; the force-distance curves from the digested molecules therefore were mainly due to the truncated C-III loop and not due to the detachment of the molecule.

The invention has been explained in detail with respect to particular embodiments and examples. Modifications and equivalents of the specific embodiments will be easily apparent to the person skilled in the art based on the disclosure of this specification. Such modifications and equivalents are intended to fall in the scope of the attached claims.

### REFERENCES

[1] Klabunde, T. and Hessler G. (2002) Chembiochem. 3: 924-44.
[2] Drake B et al. (1989) Imaging crystals, polymers, and processes in water with the atomic force microscope. Science, 243:1586-1588.
[3] Fotiadis D, Scheuring S, Muller S A, Engel A, Müller DJ. (2002) Imaging and manipulation of biological structures with the AFM. Micron, 33:385-397.
[4] Ikai A, Afrin R. (2003) Toward mechanical manipulations of cell membranes and membrane proteins using an atomic force microscope: an invited review. Cell Biochem Biophys 39:257-77.
[5] Rief M, Gautel M, Gaub H E. (2000) Unfolding forces of titin and fibronectin domains directly measured by AFM. Adv. Exp. Med. Biol. 481:129-136.
[6] M. Rief, F. Oesterhelt, B. Heymann and H. E. Gaub (1997) Science, 275, 1295-1297.
[7] Rief M, Gautel M, Oesterhelt F, Fernandez J M, Gaub H E. (1997) Reversible unfolding of individual titin immunoglobulin domains by AFM. Science 276:1109-1112.
[8] Marszalek P E, Lu H, Li H, Carrion-Vazquez M, Oberhauser A F, Schulten K, Fernandez J M. (1999) Mechanical unfolding intermediates in titin modules. Nature 402:100-103.
[9] Williams P M, Fowler S B, Best R B, Toca-Herrera J L, Scott K A, Steward A, Clarke J. (2003) Hidden complexity in the mechanical properties of titin. Nature 422:446-449.
[10] J Janovjak H, Kessler M, Gaub H, Oesterhelt D, Müller DJ. (2003) Unfolding pathways of native bacteriorhodopsin depend on temperature. EMBO J. 22:5220-5229.
[11] Müller DJ, Kessler M, Oesterhelt F, Moeller C, Oesterhelt D, Gaub H. (2002) Stability of bacteriorhodopsin alpha-helices and loops analyzed by single-molecule force spectroscopy. Biophys. J. 83:3578-3588.
[12] Cisneros D A, Oesterhelt D, Muller D J. (2005) Probing origins of molecular interactions stabilizing the membrane proteins halorhodopsin and bacteriorhodopsin. Structure (Camb) 13:235-42.
[13] Möller C, Fotiadis D, Suda K, Engel A, Kessler M, Müller DJ. (2003) Determining molecular forces that stabilize human aquaporin-1. J. Struct. Biol. 142:369-378.
[14] Kedrov A, Ziegler C, Janovjak H, Kuhlbrandt W, Müller DJ. (2004) Controlled unfolding and refolding of a single sodium-proton antiporter using atomic force microscopy. J Mol Biol 340:1143-1152.
[15] Oesterhelt F, Oesterhelt D, Pfeiffer M, Engel A, Gaub H E, Müller DJ. (2000) Unfolding pathways of individual bacteriorhodopsins. Science 288:143-146.
[16] Müller DJ, Kessler M, Oesterhelt F, Moeller C, Oesterhelt D, Gaub H. (2002) Stability of bacteriorhodopsin alpha-helices and loops analyzed by single-molecule force spectroscopy. Biophys. J. 83:3578-3588.
[17] Rief, M., Gautel, M., Oesterhelt, F., Fernandez, J. M. and Gaub, H. E. (1997) Science 276:1109-1112.
[18] Carrion-Vazquez M, Oberhauser A F, Fisher T E, Marszalek P E, Li H, Fernandez J M. (2000) Mechanical design of proteins studied by single-molecule force spectroscopy and protein engineering. Prog. Biophys. Mol. Biol. 74:63-91.
[19] Leckband D. (2000) Measuring the forces that control protein interactions. Annu. Rev. Biophys. Biomol. Struct. 29:1-26.
[20] Zhuang X, Rief M. (2003) Single-molecule folding. Curr Opin Struct Biol 13:88-97.
[21] Czajkowsky D M, Iwamoto H, Shao Z. (2000) Atomic force microscopy in structural biology: from the subcellular to the submolecular. J Electron Microsc (Tokyo) 49:395-406.
[22] Frederix P L, Akiyama T, Staufer U, Gerber C, Fotiadis D, Müller DJ, Engel A. (2003) Atomic force bio-analytics. Curr Opin Chem Biol 7:641-7.
[23] Frederix P T, Hoogenboom B, W., Fotiadis D, Müller DJ, Engel A. (2004) Atomic force microscopy of biological samples. MRS Bulletin 29:449-455.
[24] Kellermayer M S, Smith S B, Granzier H L, Bustamante C. (1997) Folding-unfolding transitions in single titin molecules characterized with laser tweezers. Science 276:1112-1, 116.
[25] Marszalek P E, Lu H, Li H, Carrion-Vazquez M, Oberhauser A F, Schulten K, Fernandez J M. (1999) Mechanical unfolding intermediates in titin modules. Nature 402:100-103.
[26] Müller DJ, Kessler M, Oesterhelt F, Moeller C, Oesterhelt D, Gaub H. (2002) Stability of bacteriorhodopsin alpha-helices and loops analyzed by single-molecule force spectroscopy. Biophys. J. 83:3578-3588.
[27] Janovjak H, Struckmeier J, Hubain M, Kedrov A, Kessler M, Muller D J. (2004) Probing the energy landscape of the membrane protein bacteriorhodopsin. Structure (Camb) 12:871-9.
[28] Assaf, S. Y. and S. H. Chung (1984) Release of endogenous Zn2+ from brain during activity. Nature 308:734-6
[29] Stojanovic A., J. Stitham, and J. Hwa (2004) Critical role of transmembrane segment zinc binding site in the structure and function of rhodopsin. J. Biol. Chem. 279, 35932-35941
[30] Ridge, K. D., Lu, Z., Liu, X. and Khorana, H. G. (1995). Structure and function in rhodopsin. Separation and characterization of the correctly folded and misfolded opsins produced on expression of an opsin mutant gene containing only the native intradiscal cysteine codons. Biochemistry 34:3261-7.
[31] Kono, M., Yu, H. and Oprian, D. D. (1998). Disulfide bond exchange in rhodopsin. Biochemistry 37:1302-5.
[32] Hwa, J., Klein-Seetharaman, J. and Khorana, H. G. (2001). Structure and function in rhodopsin: Mass spectrometric identification of the abnormal intradiscal disulfide bond in misfolded retinitis pigmentosa mutants. Proc Natl Acad Sci USA 98:4872-6.
[33] Papermaster, D. S. (1982). Preparation of retinal rod outer Segments. Methods Enzymol 81:48-52.
[34] Müller, D. J., Kessler, M., Oesterhelt, F., Moller, C., Oesterhelt, D. and Gaub, H. (2002). Stability of bacteriorhodopsin alpha-helices and loops analyzed by single-molecule force spectroscopy. Biophys J 83:3578-88.
[35] Cisneros, D. A., Oesterhelt, D. und Müller, D. J. (2005). Probing origins of molecular interactions stabilizing the membrane proteins halorhodopsin and bacteriorhodopsin. Structure (Camb) 13: 235-42.
[36] Hwa, J., Garriga, P., Liu, X. and Khorana, H. G. (1997). Structure and function in rhodopsin: packing of the helices in the transmembrane domain and folding to a tertiary structure in the intradiscal domain are coupled. Proc Natl Acad Sci USA 94:10571-6.
[37] Möller, C., Fotiadis, D., Suda, K., Engel, A., Kessler, M. and Müller, D. J. (2003) Determining molecular forces that stabilize human aquaporin-1. J Struct Biol 142:369-78.
[38] Liang, Y., Fotiadis, D., Filipek, S., Saperstein, D. A., Palczewski, K. & Engel, A. (2003). Organization of the G protein-coupled receptors rhodopsin and opsin in native membranes. Journal of Biological Chemistry 278:21655-21662.
[39] Oesterhelt, F., Oesterhelt, D., Pfeiffer, M., Engel, A., Gaub, H. E. and Müller, D. J. (2000). Unfolding pathways of individual bacteriorhodopsins. Science 288:143-6.
[40] Breitweiser, G. E. (2004] G protein-coupled receptor oligomerization: implications for G protein activation and cell signaling. Circ Res 94:17-27
[41] Garriga, P., Liu, X. and Khorana, H. G. (1996). Structure and function in rhodopsin: correct folding and misfolding in point mutants at and in proximity to the site of the retinitis pigmentosa mutation Leu-125-->Arg in the transmembrane helix C. Proc Natl Acad Sci USA 93, 4560-4.
[42] Oesterhelt, F., Oesterhelt, D., Pfeiffer, M., Engel, A., Gaub, H. E. and Müller, D. J. (2000). Unfolding pathways of individual bacteriorhodopsins. Science, 288:143-146.
[43] Müller, D. J., Kessler, M., Oesterhelt, F., Moller, C., Oesterhelt, D. and Gaub, H. (2002). Stability of bacteriorhodopsin alpha-helices and loops analyzed by single-molecule force spectroscopy. Biophys. J. 83:3578-3588.
[44] Rief, M., Gautel, M., Oesterhelt, F., Fernandez, J. M. and Gaub, H. E. (1997). Reversible unfolding of individual titin immunoglobulin domains by AFM. Science 276:1109-1112.
[45] Müller DJ, Baumeister W, Engel A. (1999) Controlled unzipping of a bacterial surface layer with atomic force microscopy. Proc. Natl. Acad. Sci. U.S.A. 96:13170-13174.
[46] Oesterhelt F, Oesterhelt D, Pfeiffer M, Engel A, Gaub H E, Müller DJ. (2000) Unfolding pathways of individual bacteriorhodopsins. Science 288:143-146.

1. A method for testing a chemical entity for its capability to modulate a (poly)peptide that is malfunctioning by means of an interaction of said chemical entity and said (poly)peptide, comprising the steps of
(a) immobilizing said malfunctioning (poly)peptide on a carrier;
(b) attaching said (poly)peptide immobilized on said carrier to a force measuring device of a force spectroscope;
(c) applying a pulling force to said (poly)peptide attached to said force measuring device and measuring the force required for stretching and/or unfolding said (poly)peptide vs. the elongation of said (poly)peptide immobilized on said carrier and attached to said force measuring device, wherein the measurements are carried out (i) prior to and (ii) after treatment of said malfunctioning polypeptide with said chemical entity;
(d) optionally comparing a force spectrum of said (poly)peptide obtained prior to said treatment and a force spectrum of said (poly)peptide obtained after said treatment, and, if the spectra are different,
(e) optionally obtaining a force spectrum of a native (poly)peptide untreated with said chemical entity that is correctly functioning and corresponds to said poly(peptide) that is malfunctioning according to steps (a), (b), and (c) (i) above;
(f) optionally comparing the force spectrum of the native (poly)peptide untreated with said chemical entity that is correctly functioning with the force spectra of said (poly)peptide obtained according to steps (c) (i) and (c) (ii) above, and
(g) optionally concluding from the difference of the force spectra of step (d) and step (f) whether said chemical entity is modulating the malfunctioning (poly)peptide towards a state of correctly functioning.

2. The method of claim 1, wherein the chemical entity is an organic chemical entity.

3. The method of claim 2, wherein the chemical entity is an inorganic entity.

4. The method of claim 1, wherein said (poly)peptide is a protein.

5. The method of claim 4, wherein said protein is selected from the group consisting of (a) a ligand-gated receptor, (b) a G-protein coupled receptor, (c) an ion channel, (d) a water channel, (e) an antiporter, (f) a communication channel, (g) a symporter, (h) a porin, (i) an ion gating channel, electrolyte gated channel and pore, glutamate gated ion channel, ATP gated channel, (j) a mechano-sensitive channel, (k) an enzyme and (l) ATP synthase.

6. The method of claim 1, wherein the (poly)peptide is (a) arranged as a three-dimensional or two-dimensional crystal, (b) bound to a biological or non-biological surface, (c) attached to a membrane or a peptide, (d) attached to a biological or synthetic molecule or (e) incorporated into a membrane.

7. The method of claim 1, wherein the (poly)peptide is embedded in a lipid bilayer.

8. A method for testing a chemical or physical entity for its capability to interact with a G protein-coupled receptor (GPCR) in its natural membrane environment, comprising the steps of
(a) immobilizing said GPCR in its natural membrane surroundings on a carrier;
(b) attaching said immobilized GPCR on said carrier to a force measuring device of a force spectroscope;
(c) applying a pulling force to said GPCR attached to said force measuring device and measuring the force required for stretching and/or unfolding said GPCR vs. the elongation of said GPCR immobilized on said carrier and attached to said force measuring device, wherein the measurements are carried out (i) prior to and (ii) after treatment of said GPCR with said chemical or physical entity;
(d) optionally comparing a force spectrum of said GPCR obtained prior to said treatment and a force spectrum of said GPCR obtained after said treatment, and
(e) optionally concluding from a difference of the force spectra of (d) whether said physical or chemical entity interacts with said GPCR.

9. The method of claim 8, wherein said difference of the force spectra of (d) allows the conclusion whether said interaction stabilizes or destabilizes said GPCR and where in said GPCR the interaction has occurred.

10. The method of claim 8, wherein said physical entity is light, voltage or current.

11. The method of claim 1, wherein said chemical entity is selected from the group consisting of a protein specific ligand, an allosteric ligand, a synthetic compound, a metallo-organic compound, a pharmaceutical compound, a toxic compound, a biochemical or biological compound, a lipid and a (poly)peptide.

12. The method of claim 1, wherein said chemical entity is selected from an electrolyte, a metal cation, a proton or hydroxyl ion and an anion.

13. The method of claim 1, wherein said carrier is (a) a biological carrier selected from the group consisting of a membrane, vesicle, cell, interface, biological structure, protein, nucleic acid molecule, fibril, fibre, and biological scaffold, or (b) an non-biological carrier selected from the group consisting of an inorganic or organic solid state material, a polymer, synthetic membrane and synthetic scaffold.

14. The method of claim 1, wherein said measuring device is selected from (a) the cantilever of an atomic force microscope, (b) magnetic tweezers, (c) optical tweezers, (d) a protein or protein complex, (e) a biological or synthetic molecule, (f) the surface of a force apparatus, (g) a membrane, and (h) the probe of a scanning probe microscope or a scanning probe spectroscope.

15. The method of claim 1, wherein said (poly)peptide or said GPCR is attached to the measuring device by adsorption.

16. The method of claim 1, wherein the pulling force applied to the (poly)peptide or GPCR is in the range of 1 to 500 pN.

17. The method of claim 1, wherein said force required for stretching and/or unfolding said (poly)peptide is recorded as a resistance of said (poly)peptide or GPCR observed upon application of said pulling force to said polypeptide.

18. The method of claim 1, wherein said force spectroscope is an atomic force microscope.

19. The method of claim 1, wherein said pulling force is exerted by said measuring device.

20. The method of claim 1, wherein said pulling force is exerted by said carrier.

21. The method of claim 1, wherein said pulling force is exerted by said (poly)peptide or said GPCR.

22. The method of claim 21, wherein said pulling force is exerted by both the measuring device and said (poly)peptide or said GPCR.

23. The method of claim 1, wherein more than one chemical entity is tested.

24. The method of claim 23, wherein said more than one chemical entity is part of a compound library.

25. The method of claim 24, wherein said screening is high-throughput screening carried out with multiple measuring devices, particularly multiple cantilevers, and/or by multiple force spectroscopes and/or by fast-speed force spectroscopy.

26. A method for testing a chemical entity for its capability to modulate a (poly)peptide that is malfunctioning by means of an interaction of said chemical entity and said (poly)peptide, said method using single-molecule force spectroscopy.

27. A method for testing a chemical or physical entity for its capability to interact with a G protein-coupled receptor (GPCR) in its natural membrane environment, said method using single-molecule force spectroscopy.
